(19) United States
(12) Patent Application Publication
(10) Pub. No.: US 2024/0059661 A1
(43) Pub. Date: Feb. 22, 2024

(54) SOLID STATE FORMS OF 2-(3,5-DICHLOROPHENYL)-1,3-BENZOXAZOLE-6-CARBOXYLIC ACID OR ITS PHARMACEUTICALLY ACCEPTABLE SALTS AND POLYMORPHS THEREOF

(71) Applicant: CIPLA LIMITED, Mumbai (IN)

(72) Inventors: Arijit DAS, Porvorim (IN); Srinivas Laxminarayan PATHI, Bangalore (IN); Ramanaiah CHENNURA, Nellore (IN); Anjaneya INDUKARI, Hyderabad (IN); Prathap RENGARAJ, Madurai (IN)

(21) Appl. No.: 18/548,691

(22) PCT Filed: Feb. 25, 2022
(86) PCT No.: PCT/IN2022/050166

§ 371 Date: Sep. 1, 2023

(30) Foreign Application Priority Data
Mar. 1, 2021 (IN) 202121008622

(51) Int. Cl. C07D 263/57

(52) U.S. Cl. CPC C07D 263/57

(57) ABSTRACT

The present disclosure relates to novel solid state forms of Tafamidis of Formula (I), and process for preparation thereof. The invention is also directed to pharmaceutical compositions containing at least one solid form and to the therapeutic or prophylactic use of such solid forms and compositions. Such compositions may be used for the treatment of transthyretin-related hereditary amyloidosis, neurodegenerative diseases, amyloidosis, neuropathic pain, amyloid fibril formation and cardiomyopathy related diseases.

### FIELD OF THE INVENTION

The present disclosure relates to novel solid state forms of Tafamidis of Formula (I). More particularly, the present invention relates to novel polymorphic forms and synergistic pharmaceutical co-crystals comprising Tafamidis and to processes of preparation thereof. The invention further relates to pharmaceutical compositions comprising novel polymorphic forms and synergistic co-crystals and at least one pharmaceutically acceptable excipient. The invention further provides novel crystalline forms of the novel pharmaceutical co-crystals.

### BACKGROUND OF THE INVENTION

The transthyretin amyloidoses (ATTR) are invariably fatal diseases characterized by progressive neuropathy and/or cardiomyopathy. ATTR are caused by aggregation of transthyretin (TTR), a natively tetrameric protein involved in the transport of thyroxine and the vitamin A-retinol-binding protein complex. Mutations within TTR that cause autosomal dominant forms of disease facilitate tetramer dissociation, monomer misfolding, and aggregation, although wild-type TTR can also form amyloid fibrils in elderly patients. Because tetramer dissociation is the rate-limiting step in TTR amyloidogenesis, targeted therapies have focused on small molecules that kinetically stabilize the tetramer, inhibiting TTR amyloid fibril formation. One such compound is Tafamidis.

2-(3,5-Dichlorophenyl)-1,3-benzoxazole-6-carboxylic acid commonly known as “Tafamidis”

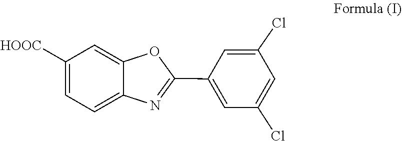 [SMILES: O=C(O)c1ccc2nc(-c3cc(Cl)cc(Cl)c3)oc2c1]

and its meglumine salts 2-(3,5-dichlorophenyl)-1,3-benzoxazole-6-carboxylic acid (1-deoxy-1-methylamino-D-glucitol (also called as “N-methyl-D-glucamine”) of formula (IA)

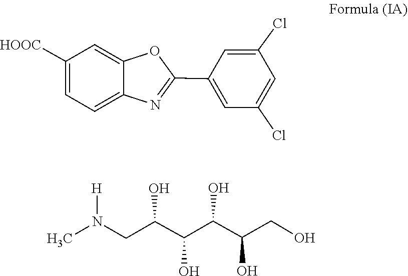 [SMILES: O=C(O)c1ccc2nc(-c3cc(Cl)cc(Cl)c3)oc2c1 ; CNC[C@H](O)[C@@H](O)[C@H](O)[C@H](O)CO]

are both approved under the brand names Vyndamax and Vyndagel respectively by USFDA on 3 May, 2019 to FoldRx Pharmaceuticals Inc. VyndaqelR and VyndamaxR both indicated for the treatment of the cardiomyopathy of wild type or hereditary transthyretin-mediated amyloidosis in adults to reduce cardiovascular mortality and cardiovascular-related hospitalization.

The drug Vyndagel was approved by the European Medicines Agency in November 2011 and by the Japanese Pharmaceuticals and Medical Devices Agency in September 2013 for the treatment of Transthyretin Type Familial Amyloid Polyneuropathy (TTR-FAP) and demonstrated a slowing of disease progression in patients heterozygous for the V30M TTR mutation.

VyndaqelR available in 20 mg soft gelatin capsules, each soft capsule contains 20 mg of micronized Tafamidis meglumine equivalent to 12.2 mg Tafamidis. VyndamaxR available in 61 mg of Tafamidis soft gelatin capsule orally once daily.

U.S. Pat. No. 7,214,695 B2 discloses Tafamidis and its pharmaceutically acceptable salts and also process for the preparation.

U.S. Pat. No. 9,2491,12 B2 discloses crystalline form M, liquid crystal form B and amorphous form A of Tafamidis meglumine. This patent also discloses a process for the preparation of amorphous form of Tafamidis meglumine by melting.

U.S. Pat. No. 9,770,441 B1 discloses crystalline Form 1, Form 2, Form 4, Form 6 and amorphous form of Tafamidis of formula (I) and its preparation.

US 20190119226 A1 discloses crystalline Form E of Tafamidis meglumine and process for its preparation thereof.

WO 2020/232325 patent application publication discloses crystalline Forms I, II, III, IV and V of Tafamidis and process for its preparation thereof.

WO 2021/001858 patent application publication discloses crystalline Forms S, N and R of Tafamidis and amorphous premix of Tafamidis meglumine of formula (IA) with one or more pharmaceutically acceptable excipients and process for its preparation thereof.

The pharmaceutical industry is often confronted with the phenomenon of multiple polymorphs of the same crystalline chemical entity. Polymorphism is often characterized as the ability of a drug substance to exist as two or more crystalline phases that have different arrangements and/or conformations of the molecules in the crystal lattices giving the crystals different physicochemical properties.

Multiple crystal forms with different solid state properties of a drug substance can exhibit differences in storage, stability, compressibility, density and dissolution rates (important in determining bioavailability) during processing. Powder X-ray Diffraction is a powerful tool in identifying different crystal phases by their unique diffraction patterns.

Regulatory agencies worldwide require a reasonable effort to identify the polymorphs of the drug substance and check for polymorph interconversions. Due to the often-unpredictable behavior of polymorphs and their respective differences in physicochemical properties, consistency in manufacturing between batches of the same product must be demonstrated.

The ability to be able to manufacture the selected polymorphic form reliably is a key factor in determining the success of the drug product. Proper understanding of the polymorph landscape and nature of the polymorphs of a pharmaceutical will contribute to manufacturing consistency.

The discovery of new polymorphic forms, new salts, solid state forms, and solvates of a pharmaceutically useful compound provides a new opportunity to improve the performance characteristics of a pharmaceutical product such as stability, solubility, bioavailability, etc. It enlarges the repertoire of materials that a formulation scientist has available for designing, for example, a pharmaceutical dosage form of a drug with a targeted release profile, a different crystal habit, higher crystallinity, or polymorphic stability, or solubility, which may offer better processing or handling characteristics, improved dissolution profile, or improved shelf-life.

Different new salts, solid state forms, and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Therefore, there is a continuous need in the art to develop stable and soluble solid state forms of Tafamidis, which are having greater stability, flowability, dissolution properties; thereby increasing the bioavailability as well as to enhance the efficacy, of the parent molecule in lower doses.

### OBJECTIVES OF THE INVENTION

An object of the present invention is to provide novel solid state forms of Tafamidis such as novel crystalline forms and co-crystals.

Another object of the present invention is to provide a process for the preparation of novel solid state forms of Tafamidis.

Yet another object of the invention is to provide pharmaceutical composition comprising a therapeutically effective amount of novel solid state forms of Tafamidis.

Yet another object of the invention is to provide method of treatment of the cardiomyopathy of wild type or hereditary transthyretin-mediated amyloidosis, wherein novel solid state forms of Tafamidis, are useful.

### SUMMARY OF THE INVENTION

In accordance with the above objective, the present invention is directed to novel solid state forms Tafamidis and salts thereof, methods of preparing such solid state forms, and methods of treating transthyretin-mediated amyloidosis with such novel solid state forms. The novel solid state forms may be a crystalline polymorph or a co-crystal.

In a first aspect, the present invention provides novel crystalline forms of Tafamidis, hereinafter referred to as Form C1, Form C2, Form C3, Form C4, Form C5 and Form C6.

The polymorphic forms of Tafamidis may be in a pseudo polymorphic form. Accordingly, pseudo polymorphs are provided that include hydrates and/or solvates.

The crystalline nature of forms according to the present invention is characterized by X-ray powder diffraction.

In a second aspect, the present invention relates to processes for preparing novel polymorphic forms of Tafamidis thereof.

In a third aspect, the present invention relates novel co-crystals of Tafamidis.

A “co-crystal” according to the present invention is a single chemical entity comprising two or more different elements that have a unique and defined chemical structure. A “co-crystal” consist of a fixed ratio of atoms that are held together in a defined spatial arrangement by ionic, covalent, hydrogen bonds, van der Waals forces or 7E- 7E interactions. According to the present invention the elements of a “co-crystal” comprise Tafamidis and an alkaloid component, water, ions, solvents, or co-formers.

In addition, a “co-crystal” according to the present invention represent “a druggable form” of a Tafamidis with an alkaloid component. A “druggable form” as used herein is defined as any form (salt, amorphous, crystal (of a salt), co-crystal, solution, dispersion, mixture, etc.) that Tafamidis with an alkaloid component might take which still can be formulated into a pharmaceutical formulation usable as a medicament to treat a disease or a symptom.

Accordingly, the present invention provides novel synergistic pharmaceutical compounds of Tafamidis with an alkaloid component also recalled as “co-former”.

The novel pharmaceutical compounds are relatively stable towards the moisture and humidity, thereby representing an amorphous or a crystalline form of pharmaceutical compound, thus enhancing the efficacy of the parent molecule in lower doses.

In a fourth aspect, the present invention provides a novel co-crystal of Tafamidis and nicotinamide.

In a fifth aspect, the present invention provides a novel co-crystal of Tafamidis and caffeine.

The novel co-crystals of Tafamidis are relatively stable towards the moisture and humidity, thereby representing an amorphous or a crystalline form of pharmaceutical compound, thus enhancing the efficacy of the parent molecule in lower doses.

The novel co-crystals of the present invention could be either in a crystalline or amorphous form.

The crystalline nature of co-crystals according to the present invention is characterized by X-ray powder diffraction.

In a sixth aspect, the present invention relates to processes for preparing novel co-crystals of Tafamidis thereof.

In a seventh aspect, the present invention provides a pharmaceutical composition comprising novel crystalline forms and co-crystal of Tafamidis and processes for the preparation of the novel composition.

In an eight aspect, the present invention provides use of the novel crystalline forms and co-crystals of Tafamidis for the preparation of a pharmaceutical composition and to a pharmaceutical composition comprising an effective amount of the novel crystalline forms and co-crystals of Tafamidis of the present invention and at least one pharmaceutically acceptable excipient. Such pharmaceutical composition may be administered to a mammalian patient in any dosage form, e.g. solid, liquid, powder, elixir, injectable solution, etc.

In a nineth aspect, the present invention is directed to methods of treating and/or preventing transthyretin-mediated amyloidosis by administering a therapeutically effective amount of a novel crystalline forms or co-crystal of Tafamidis thereof.

In a tenth aspect, the present invention is directed to the use of novel crystalline forms or co-crystal of Tafamidis in the manufacture of a medicament for treating and/or preventing transthyretin-mediated amyloidosis.

### BRIEF DESCRIPTION OF THE FIGURES

FIG. 1 shows an X-ray powder diffraction pattern (“PXRD”) of Tafamidis Form C1.

FIG. 2 shows a Thermogravimetric Analysis curve (“TGA”) of Tafamidis Form C1.

FIG. 3 shows a Differential Scanning calorimetry (“DSC”) thermogram of Tafamidis Form C1.

FIG. 4 shows an X-ray powder diffraction pattern of Tafamidis Form C2.

FIG. 5 shows a Thermogravimetric Analysis curve of Tafamidis Form C2.

FIG. 6 shows a Differential Scanning calorimetry thermogram of Tafamidis Form C2.

FIG. 7 shows an X-ray powder diffraction pattern of Tafamidis Form C3.

FIG. 8 shows a Thermogravimetric Analysis curve of Tafamidis Form C3.

FIG. 9 shows a Differential Scanning calorimetry thermogram of Tafamidis Form C3.

FIG. 10 shows an X-ray powder diffraction pattern of Tafamidis Form C4.

FIG. 11 shows a Thermogravimetric Analysis curve of Tafamidis Form C4.

FIG. 12 shows a Differential Scanning calorimetry thermogram of Tafamidis Form C4.

FIG. 13 shows an X-ray powder diffraction pattern of Tafamidis Form C5.

FIG. 14 shows a Thermogravimetric Analysis curve of Tafamidis Form C5.

FIG. 15 shows a Differential Scanning calorimetry thermogram of Tafamidis Form C5.

FIG. 16 shows an X-ray powder diffraction pattern of Tafamidis Form C6.

FIG. 17 shows a Thermogravimetric Analysis curve of Tafamidis Form C6.

FIG. 18 shows a Differential Scanning calorimetry thermogram of Tafamidis Form C6.

FIG. 19 shows an X-ray powder diffraction pattern of Tafamidis Nicotinamide co-crystal.

FIG. 20 shows an X-ray powder diffraction pattern of Tafamidis Caffeine co-crystal.

FIG. 21 shows an X-ray powder diffraction pattern of Tafamidis Form C1 as per example 10.

FIG. 22 shows a Differential Scanning calorimetry thermogram of Tafamidis Form C1 as per example 10.

FIG. 23 shows a Thermogravimetric Analysis curve of Tafamidis Form C1 as per example 10.

FIG. 24 shows an X-ray powder diffraction pattern of Tafamidis Form C1 as per example 11.

FIG. 25 shows a Differential Scanning calorimetry thermogram of Tafamidis Form C1 as per example 11.

FIG. 26 shows a Thermogravimetric Analysis curve of Tafamidis Form C1 as per example 11.

### DETAILED DESCRIPTION

The invention will now be described in detail in connection with certain preferred and optional embodiments, so that various aspects thereof may be more fully understood and appreciated.

As used herein, the term “solvated” is understood to mean formation of a complex of variable stoichiometry comprising Tafamidis of Formula (I) and a solvent. Such solvents for the purpose of the invention may not interfere with the biological activity of the solute. Typically, the solvent used is a pharmaceutically acceptable solvent. Examples of suitable pharmaceutically acceptable solvents include but not limited to C1-C4 alcohol solvents, MDC, acetone, methyl benzoate, ethyl acetate, benzyl alcohol, 4-methyl pentane 2-one, propylene glycol, acetonitrile, 1,3-dioxane, and dimethylsulfoxide (DMSO), and solvates of more than 1%.

The solvate can be isolated either as an amorphous form or in a crystalline form, preferably in crystalline form.

The solvate can be further isolated either in anhydrous form or hydrated form.

As used herein, the term “hydrate” is understood as a substance that is formed by adding water molecules. The skilled person will appreciate that the water molecules are absorbed, adsorbed or contained within a crystal lattice of the solid compounds, usually in defined stoichiometric ratio. The notation for a hydrated compound may be ·nH₂O, where n is the number of water molecules per formula unit of the compound. For example, in a hemihydrate, n is 0.5; in a monohydrate n is one; in a sesquihydrate, n is 1.5; in a dihydrate, n is 2; and so on.

In comparison to the restricted stoichiometric hydrates, non-stoichiometric hydrates can vary in water content without major change in their crystal structure. The amount of water in the crystal lattice only depends on the partial pressure of water in the surrounding atmosphere.

Structurally, non-stoichiometric hydrates normally show channels or networks, through which the water molecules can diffuse. Depending on how the water is arranged inside the crystals, they are classified as isolated hydrates, channel hydrates and ion associated hydrates.

As used herein, the term “substantially the same X-ray powder diffraction pattern” is understood to mean that those X-ray powder diffraction patterns having diffraction peaks with 2θ values within ±0.2° of the diffraction pattern referred to herein are within the scope of the referred to diffraction pattern.

In one aspect the present invention provides novel polymorphic forms of the transthyretin stabilizer Tafamidis of Formula (I). The Tafamidis may be isolated in pseudo polymorphic form as a solvate optionally in hydrated form, or as a non-hydrated solvate.

Accordingly, pseudo polymorphs provided include solvates, more in particular, C1-C4 alcohol solvates, a MDC solvate, an acetone solvate, a benzyl alcohol solvate, an ethyl acetate solvate, a 4-methyl pentane 2-one solvate, a propylene glycol solvate, an acetonitrile solvate, a 1,3-dioxane solvate, and a dimethyl sulfoxide (DMSO) solvate, optionally in hydrated form.

As polymorphic forms are reliably characterized by peak positions in the X-ray diffractogram, the polymorphs of the present invention have been characterized by powder X-ray diffraction spectroscopy which produces a fingerprint of the particular crystalline form. Measurements of 2θ values are accurate to within ±0.2 degrees. All the powder diffraction patterns were measured on a Rigaku Dmax 2200 advanced X-ray powder diffractometer with a copper-K-α radiation source.

Thus, in first aspect, the present invention provides the crystalline Tafamidis, which is herein and in the claims designated as “Form C1”, which has good flow characteristics.

The crystalline Form C1 is relatively stable towards moisture and humidity, thereby representing a crystalline form of Tafamidis, thus enhancing the efficacy of the parent molecule in lower doses.

Crystalline Form C1 of Tafamidis may be an anhydrous form or a solvated form.

The crystalline Form C1 according to the present invention may be characterized by powder X-ray diffraction.

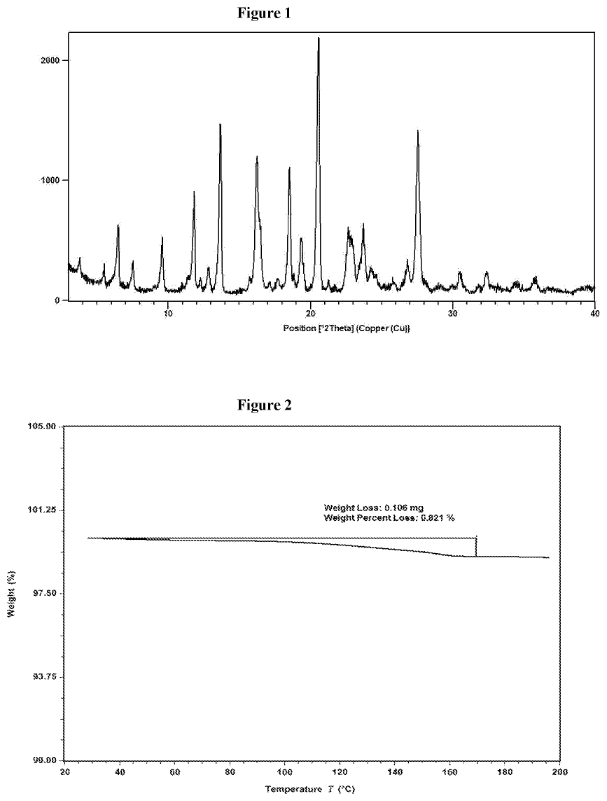

The Crystalline Form C1 may be characterized by having an XRPD diffractogram comprising peaks at 5.48, 6.44, 7.51, 9.57, 11.80, 13.65, 18.51 and 20.53±0.2 °2θ. The XRPD diffractogram may comprise further peaks at 16.24, 19.36, 23.68 and 27.55±0.2 °2θ. The XRPD diffractogram may be as depicted in FIG. 1.

In an embodiment, crystalline Form C1 of Tafamidis is characterized by having a thermogravimetric analysis as shown in FIG. 2.

TGA data indicated a weight loss of 0.8% at temperatures up to 170° C. The TGA analysis indicates the crystalline Form C1 of Tafamidis is the anhydrous form.

The crystalline Form C1 of Tafamidis may also be characterized as having a DSC thermogram exhibiting a first small endotherm peak with an onset at around 152.63 ±5° C. and a peak maximum at around 161.44±5° C. and a second melting endothermic peak with an onset melting point around 288.48±5° C. and a peak maximum at around 288.5±5° C.

In an embodiment, crystalline Form C1 of Tafamidis may is characterized by having a DSC thermogram as shown in FIG. 3.

Optionally, the crystalline Form C1 of Tafamidis may be further characterized by data selected from the group consisting of: an X-ray powder diffraction pattern having peaks at about 5.48, 6.44, 7.51, 9.57, 11.80, 13.65, 18.51 and 20.53±0.2 °2θ; an X-ray powder diffraction pattern having peaks at about 16.24, 19.36, 23.68 and 27.55±0.2 °2θ; a XRPD diffractogram as depicted in FIG. 1; a DSC thermogram having a first endothermic peak in the range of about 161.44±5° C. and a second endothermic peak in the range of about 288.5±5° C.; a DSC pattern as depicted in FIG. 3; a TGA pattern as depicted in FIG. 2; and combinations thereof.

Those skilled in the art would recognize that polymorphic forms of the present invention may be further characterized by a variety of other solid state spectroscopic techniques including, but not limited to, Raman spectroscopy, FTIR spectroscopy, vibrational spectroscopy, polarized light microscopy (PLM), and solid state NMR, the ¹³C NMR and ¹H NMR (in a suitable solvent, e.g., in D₂O or DMSO-i¾) to evaluate the chemical structure, Dynamic Gravimetric Vapor Sorption (DVS) to evaluate the hygroscopicity, hot-stage optical microscopy to examine thermal transitions and/or chromatography (e.g., HPLC) in a suitable solvent to evaluate the purity. Products as described herein can be further analysed via Karl Fischer Titration (KF) to determine the water content.

Preferably the crystalline Form C1 of Tafamidis, has a crystalline purity of at least 80%, more preferably at least 90%, more preferably at least 95%, most preferably at least 99% by weight.

### Solid State Stability

Forced degradation is synonymous with stress testing and purposeful degradation. Purposeful degradation can be a useful tool to predict the stability of a drug substance or a drug product with effects on purity, potency, and safety. It is imperative to know the impurity profile and behavior of a drug substance under various stress conditions. Forced degradation also plays an important role in the development of analytical methods, setting specifications, and design of formulations under the quality-by-design (QbD) paradigm. The nature of the stress testing depends on the individual drug substance and the type of drug product (e.g., solid oral dosage, lyophilized powders, and liquid formulations) involved.

A forced degradation study was performed to generate potential degradation products and assess stability liabilities. Tafamidis form C1 prepared as per the present disclosure was subjected to forced degradation study with below specified conditions. After exposure for 7 days at room temperature, the samples were further investigated at 40° C./75% RH using open and closed cups for 7 days. The samples were analysed at pre-determined time intervals for PXRD, HPLC purity and water content. The data indicate that there is no significant change with respect to PXRD, HPLC purity and water content of Tafamidis form C1. The data did not show any degradation under any of the stress conditions. The forced degradation results for Tafamidis Form C1 are tabulated below in Table 1.

TABLE 1
Conditions for Forced Degradation Studies

| Sampling | | Form C1 | | | |
|---|---|---|---|---|---|
| Time | Storage | | | Water | HPLC |
| (days) | Condition | Description | PXRD | content KF | Purity |
| 0 D | Initial | off-white powder | Form C1 | 0.57 | 99.98 |
| 7 D | 25° C./60% RH | off-white powder | Comparable with | 0.48 | 99.98 |
| | Open cup | | initial | | |
| 7 D | 25° C./60% RH | off-white powder | Comparable with | 0.42 | 99.98 |
| | Closed cup | | initial | | |
| 7 D | 40° C./75% RH | off-white powder | Comparable with | 0.72 | 99.98 |
| | Open cup | | initial | | |
| 7 D | 40° C./75% RH | off-white powder | Comparable with | 0.45 | 99.98 |
| | Closed cup | | initial | | |
| | 10 TORR | off-white powder | Comparable with | Not | 99.99 |
| | | | initial | Performed | |
| 5 D | 60°C/80% RH | off-white powder | Comparable with | 0.62 | 99.97 |
| | | | initial | | |
| 6 Hours | 105° C. | off-white powder | Comparable with | Not | 99.98 |
| (DHS) | | | initial | performed | |
| 6 Hours | 120° C. | off-white powder | Comparable with | Not | 99.99 |
| | | | initial | performed | |

### Indicative Stability

The stability of Tafamidis form C1 prepared as per the present disclosure was studied by storing the samples at 2-8° C., 25° C./60% RH; and 40° C./75% RH storage conditions upto 6 months. The samples were analysed for PXRD, HPLC purity and water content at pre-determined time intervals of 1M, 2M, 3M and 6M.

The data indicates that there is no significant change with respect to PXRD, HPLC purity and water content in all the storage conditions up to 6 months. The stability data collected after 6 months of storage are tabulated below in Table 2.

TABLE 2
Indicative Stability of Polymorph Form C1 of Tafamidis

| Sampling | | | Water | | |
|---|---|---|---|---|---|
| Time | Storage | | content | | HPLC |
| (days/months) | Condition | Description | KF | PXRD | Purity |
| 0 | Initial | Off-white colour | 0.34 | Form C1 | 99.99 |
| | | powder | | | |
| 1 month | 2-8° C. | Off-white colour | 0.88 | Comparable with | 99.98 |
| | | powder | | initial | |
| 2 months | 2-8° C. | Off-white colour | 0.63 | Comparable with | 99.98 |
| | | powder | | initial | |
| 3 months | 2-8° C. | Off-white colour | 1.02 | Comparable with | 99.98 |
| | | powder | | initial | |
| 6 months | 2-8° C. | Off-white colour | 0.79 | Comparable with | 99.98 |
| | | powder | | initial | |
| 1 month | 25° C./60% RH | Off-white colour | 0.85 | Comparable with | 99.98 |
| | | powder | | initial | |
| 2 months | 25° C./60% RH | Off-white colour | 0.68 | Comparable with | 99.98 |
| | | powder | | initial | |
| 3 months | 25° C./60% RH | Off-white colour | 0.92 | Comparable with | 99.98 |
| | | powder | | initial | |
| 6 months | 25° C./60% RH | Off-white colour | 0.61 | Comparable with | 99.98 |
| | | powder | | initial | |
| 1 month | 40° C./75% RH | Off-white colour | 0.80 | Comparable with | 99.99 |
| | | powder | | initial | |
| 2 months | 40° C./75% RH | Off-white colour | 0.78 | Comparable with | 99.98 |
| | | powder | | initial | |
| 3 months | 40° C./75% RH | Off-white colour | 0.85 | Comparable with | 99.98 |
| | | powder | | initial | |
| 6 months | 40° C./75% RH | Off-white colour | 0.65 | Comparable with | 99.98 |
| | | powder | | initial | |

The invention encompasses a process for preparing the crystalline Form C1 of Tafamidis.

In one embodiment process comprises, slurrying Tafamidis in a suitable non polar solvent; stirring for sufficient time, isolating the solid and drying the solid.

Tafamidis used for the above process, as well as for the following processes, may be in any polymorphic form or in a mixture of any polymorphic forms such as hydrated, solvated, non-solvated or mixture of hydrated, solvated or non-solvated forms thereof. The Tafamidis used in the processes of the present invention can be obtained by any method known in the art, such as the one described in the U.S. Pat. No. 7,214,695 B2.

Preferably, Tafamidis used is Tafamidis formic acid solvate.

Preferably, solvent is halogenated solvent selected from the group comprising of dichloromethane, dichloroethane, chloroform and the like.

Preferably before mixing solvent is chilled to −20 to −15° C.

Optionally, following the chilling step, a seeding with Tafamidis is performed to obtain slurry of crystalline Form C1 of Tafamidis. Preferably, the seeding is done with Tafamidis Form C1.

Preferably, the process further comprises a first stirring step. Preferably, the first stirring is for about 4 hours to about 6 hours. Preferably, the stirring is at about −20° C. to about −15° C.

Preferably, the process further comprises a second stirring step. Preferably, the second stirring is for about 4 hours to about 6 hours. Preferably, the stirring is at about 0° C. to about 10° C.

Preferably, the process further comprises a third stirring step. Preferably, the third stirring is for about 6 hours to about 12 hours. Preferably, the stirring is at about 25° C. to about 30° C.

Preferably, the obtained solid form is isolated. Preferably, the isolation is done by centrifugation.

The drying may be done in a vacuum oven at a temperature of about 35° C. to about 45° C. for about 1 hour to about 5 hours.

Preferably, the obtained solid is further slurried in a polar aprotic solvent selected from the group comprising of acetone, acetonitrile, THF, methyl acetate, isopropyl acetate, isobutyl acetate; non-polar solvents like hexane, heptane, pentane or protic solvents like isopropanol, 1-butanol and the like.

Preferably, polar aprotic solvent is acetone.

Preferably, the process further comprises a stirring step. Preferably, stirring is for about 1 hour to about 10 hours, more preferably, for about 3 hours to about 5 hours. Preferably, the stirring is at about −15° C. to about −5° C., more preferably, at about −12° C. to about −10° C.

Preferably, the obtained solid form is isolated. Preferably, the isolation is done by filtration.

The drying may be done in a vacuum oven at a temperature of about 50° C. to about 70° C.

Preferably, the obtained solid is dried under reduced pressure at 50° C. to about 80° C., preferably at 60° C. to about 70° C., for a period of about 20 hours to about 3 days, more preferably for about 1 day to about 2 days.

In second embodiment process comprises, slurrying Tafamidis salt in a suitable mixture of non polar solvent and acid, stirring for sufficient time, isolating the solid and drying the solid.

Preferably, Tafamidis salt used is Tafamidis meglumine.

Tafamidis meglumine used for the above process, as well as for the following processes, may be in any polymorphic form or in a mixture of any polymorphic forms such as hydrated, solvated, non-solvated or mixture of hydrated, solvated or non-solvated forms thereof.

Preferably, non polar solvent is halogenated solvent selected from the group comprising of dichloromethane, dichloroethane, chloroform and the like.

Preferably, acid used is aqueous hydrochloric acid.

Preferably solvent mixture is chilled to −10 to 0° C.

Preferably, the process further comprises a stirring step. Preferably, stirring is for about 2 hours to about 10 hours, more preferably, for about 3 hours to about 5 hours. Preferably, the stirring is at about −10° C. to about 10° C., more preferably, at about 0° C. to about 5° C.

Preferably, the obtained solid form is isolated. Preferably, the isolation is done by filtration.

Preferably, the obtained solid is further slurried in a polar solvent.

Preferably, polar solvent is water.

Preferably, the process further comprises a stirring step. Preferably, stirring is for about 30 minutes to about 5 hours, more preferably, for about 1 hour to about 2 hours. Preferably, the stirring is at about −5° C. to about 5° C., more preferably, at about 0° C. to about 5° C.

Preferably, the obtained solid form is isolated. Preferably, the isolation is done by filtration.

The drying may be done in a vacuum oven at a temperature of about 50° C. to about 70° C.

Preferably, the obtained solid is dried under reduced pressure at 20° C. to about 50° C., preferably at 30° C. to about 40° C., for a period of about 30 minutes to about 5 hours, more preferably for about 1 hour to about 3 hours.

In a third embodiment process comprises, treating Tafamidis with a suitable base in a suitable non polar solvent; treating Tafamidis salt solution with IPA-HCl and isolating the precipitated solid Tafamidis free acid and drying the solid, wherein the process is carried out without isolating Tafamidis salt.

A suitable base used for the reaction may be an inorganic or organic base. The inorganic base may be selected from the group comprising of alkali or alkaline earth metal carbonates, such as cesium carbonate, sodium carbonate, potassium carbonate, magnesium carbonate, calcium carbonate, lithium carbonate or barium carbonate; alkali or alkaline earth metal hydroxides such as sodium hydroxide, potassium hydroxide, lithium hydroxide, magnesium hydroxide, calcium hydroxide, strontium hydroxide or barium hydroxide; amino acids such as arginine, lysine.

Organic bases may be aliphatic or aromatic and may be selected from, but not limited to ethyl amine, diethyl amine, triethyl amine, triethanolamine, Di isopropyl amine, N,N-diisopropylethylamine, meglumine, N-methyl-D-glucamine, phenyl ethyl amine and isomers thereof, naphthyl ethyl amine and isomers thereof, pyridine, picoline, piperidine, dicyclohexylamine. or cyclohexyl amine.

Preferably, solvent is selected from halogenated hydrocarbons solvent selected from the group comprising of dichloromethane, dichloroethane, chloroform and the like.

Preferably, the Tafamidis salt is not isolated. Optionally, the Tafamidis salt is isolated.

Preferably, the Tafamidis salt solution is treated with charcoal.

Preferably, clear filtrate is treated with IPA-HCl solution at about 20° C. to about 30° C.

Optionally, a seeding with Tafamidis is performed to obtain crystalline Form C1 of Tafamidis. Preferably, the seeding is done with Tafamidis Form C1.

Preferably, the process further comprises a first stirring step. Preferably, the first stirring is for about 24 hours to about 48 hours. Preferably, the stirring is done at about 20° C. to about 30° C.

Preferably, the obtained solid form is isolated. Preferably, the isolation is done by centrifugation.

Preferably, the obtained solid is further slurried in a halogenated hydrocarbons solvent selected from dichloromethane, dichloroethane, and chloroform. Preferably, halogenated hydrocarbons solvent is dichloromethane.

Preferably, the process further comprises a stirring step. Preferably, stirring is for about 30 minutes to about 2 hours. Preferably, the stirring is at about 15° C. to about 30° C., more preferably, at about 20° C. to about 25° C.

Preferably, the obtained solid form is isolated. Preferably, the isolation is done by centrifugation.

Optionally, the process further comprises a second slurry step. Preferably, the second slurring is done in a polar aprotic solvent selected from acetone, acetonitrile, THF, ethyl acetate, methyl acetate, Isopropyl acetate, Isobutyl acetate or non-polar solvents heptane, Pentane or protic solvents like Isopropanol, 1-butanol etc. and the like.

Preferably, polar aprotic solvent is acetone.

Preferably, the process further comprises a stirring step. Preferably, stirring is for about 15 minutes to about 2 hours, more preferably, for about 30 minutes to about 1 hour. Preferably, the stirring is at about −10° C. to about 10° C., more preferably, at about −5° C. to about 5° C.

Preferably, the obtained solid form is isolated. Preferably, the isolation is done by filtration.

The drying may be done in a vacuum oven.

Preferably, the obtained solid is dried under reduced pressure at 30° C. to about 90° C., preferably at 40° C. to about 90° C., for a period of about 3 hours to about 30 hours, more preferably for about 4 hours to about 25 hours.

Optionally, the dried solid may be further milled to obtain desired particle size and may be optionally dried further in a vacuum oven.

The drying may be done in a vacuum oven at a temperature of about 30° C. to about 60° C. for about 2 hours to about 40 hours, more preferably for about 3 hours to about 30 hours.

The Form C1 of Tafamidis, obtained per the present invention is substantially free from other forms of Tafamidis. “Substantially free” from other forms of Tafamidis shall be understood to mean that the polymorphs of Tafamidis contain less than 10%, preferably less than 5%, of any other forms of Tafamidis and less than 1% of other impurities.

According to a second aspect, the present invention provides the crystalline Tafamidis, which is herein and in the claims designated as “Form C2”, which has good flow characteristics.

The crystalline Form C2 is relatively stable towards moisture and humidity, thereby representing a crystalline form of Tafamidis, thus enhancing the efficacy of the parent molecule in lower doses.

Crystalline Form C2 of Tafamidis may be an anhydrous form or a solvated form. In specific embodiments, Form C2 of Tafamidis may be an anhydrous form or an ethanol solvate.

The crystalline Form C2 according to the present invention may be characterized by powder X-ray diffraction.

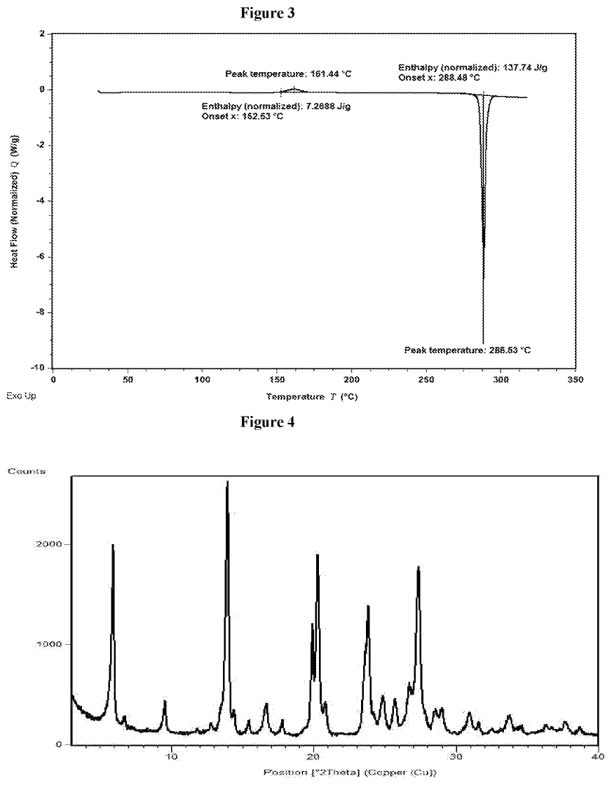

The Crystalline Form C2 may be characterized by having an XRPD diffractogram comprising peaks at 5.89, 9.52, 13.91, 16.62, 19.88, 20.27, and 27.32 ±0.2 °2θ. The XRPD diffractogram may be as depicted in FIG. 4.

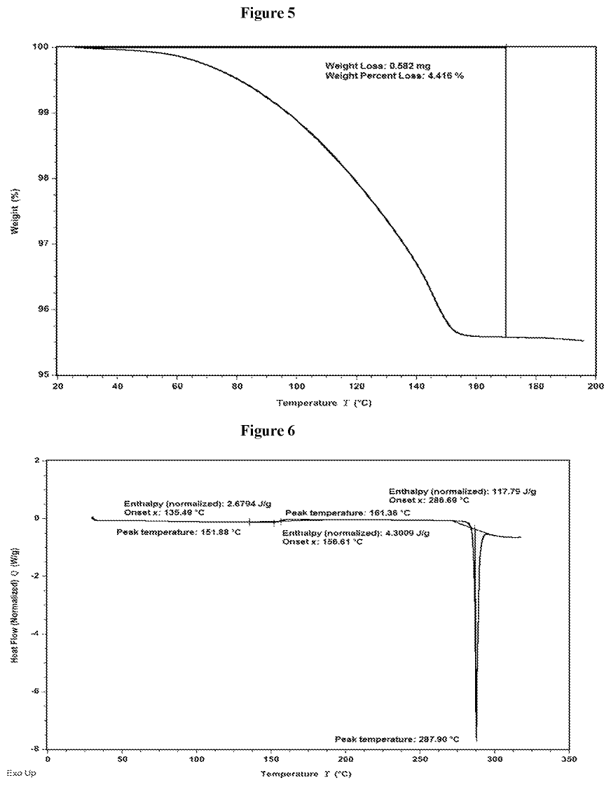

In an embodiment, crystalline Form C2 of Tafamidis is characterized by having a thermogravimetric analysis as shown in FIG. 5.

TGA data indicated a weight loss of 4.4% at temperatures up to 170° C. The TGA analysis indicates the crystalline Form C2 of Tafamidis is the ethanol solvate form.

The crystalline Form C2 of Tafamidis may also be characterized as having a DSC thermogram exhibiting a first small endothermic peak with an onset at around 135.49±5° C. and a peak maximum at 151.88° C.±5° C.; a second endothermic peak with an onset at around 156.61° C.±5° C. and a peak maximum at 161.36±5° C. and a third melting endothermic peak with an onset at around 286.7±5° C. and a peak maximum at around 287.9±5° C.

In an embodiment, crystalline Form C2 of Tafamidis may is characterized by having a DSC thermogram as shown in FIG. 6.

Optionally, the crystalline Form C2 of Tafamidis may be further characterized by data selected from the group consisting of: an X-ray powder diffraction pattern having peaks at about 5.89, 9.52, 13.91, 16.62, 19.88, 20.27, and 27.32±0.2 °2θ; a XRPD diffractogram as depicted in FIG. 4; a TGA pattern as depicted in FIG. 5; a DSC thermogram having a first small endothermic peak in the range of about 151.88° C.±5° C. , a second endothermic peak in the range of about 161.36±5° C. and a third endothermic peak in the range of about 287.9±5° C.; a DSC pattern as depicted in FIG. 6; and combinations thereof.

Preferably the crystalline Form C2 of Tafamidis, has a crystalline purity of at least 80%, more preferably at least 90%, more preferably at least 95%, most preferably at least 99% by weight.

The invention encompasses a process for preparing the crystalline Form C2 of Tafamidis

In an embodiment process comprises, slurrying Tafamidis in a suitable first solvent; adding second solvent; isolating the precipitated solid and drying the solid.

Preferably, Tafamidis used is Tafamidis formic acid solvate.

Preferably, first solvent is a polar solvent selected from C1-C5 alcoholic solvent, more preferably ethanol. Preferably, solvent is chilled to −20 to −15° C.

Optionally, following the chilling step, a seeding with Tafamidis is performed to obtain crystalline Form C2 of Tafamidis. Preferably, the seeding is done with Tafamidis Form C2.

Preferably, the process further comprises a stirring step. Preferably, the stirring is for about 4 hours to about 6 hours. Preferably, the stirring is at about −20° C. to about −15° C.

Preferably, second solvent is a non polar solvent, more preferably halogenated solvent, most preferably selected from n-Heptane and n-Hexane.

Preferably, prior to mixing second solvent is prechilled to about −20° C. to about −15° C.

Preferably, the process further comprises a second stirring step. Preferably, the second stirring is for about 30 minutes to about 2 hours. Preferably, the stirring is at about −20° C. to about −15° C.

Preferably, the obtained solid form is isolated. Preferably, the isolation is done by centrifugation.

The drying may be done in a vacuum oven at a temperature of about 35° C. to about 45° C. for about 1 hour to about 5 hours.

The Form C2 of Tafamidis, obtained per the present invention is substantially free from other forms of Tafamidis. “Substantially free” from other forms of Tafamidis shall be understood to mean that the polymorphs of Tafamidis contain less than 10%, preferably less than 5%, of any other forms of Tafamidis and less than 1% of other impurities.

According to a third aspect, the present invention provides the crystalline Tafamidis, which is herein and in the claims designated as “Form C3”, which has good flow characteristics.

The crystalline Form C3 is relatively stable towards moisture and humidity, thereby representing a crystalline form of Tafamidis, thus enhancing the efficacy of the parent molecule in lower doses.

Crystalline Form C3 of Tafamidis may be an anhydrous form or a solvated form. In specific embodiments, Form C3 of Tafamidis may be an anhydrous form or an acetone solvate.

The crystalline Form C3 according to the present invention may be characterized by powder X-ray diffraction.

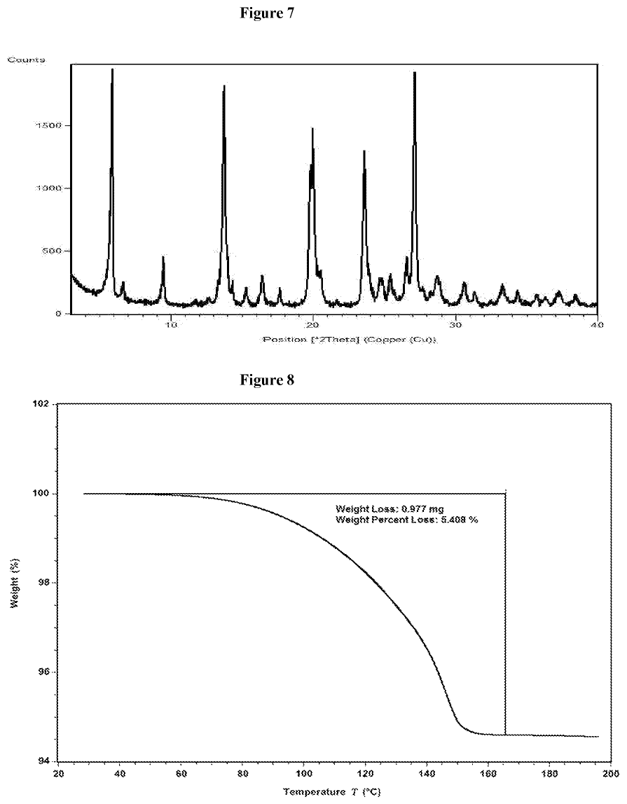

The Crystalline Form C3 may be characterized by having an XRPD diffractogram comprising peaks at 5.89, 9.48, 13.74, 16.43 and 23.62±0.2 °2θ. The XRPD diffractogram may be as depicted in FIG. 7.

In an embodiment, crystalline Form C3 of Tafamidis is characterized by having a thermogravimetric analysis as shown in FIG. 8.

TGA data indicated a weight loss of 5.4% at temperatures up to 165° C. The TGA analysis indicates the crystalline Form C3 of Tafamidis is the acetone solvate form.

The crystalline Form C3 of Tafamidis may also be characterized as having a DSC thermogram exhibiting a first small endothermic peak with an onset at around 148.23±5° C. and a peak maximum at 154.08° C.±5° C.; a second endothermic peak with an onset at around 157.08° C.±5° C. and a peak maximum at 160.9±5° C. and a third melting endothermic peak with an onset at around 286.6±5° C. and a peak maximum at around 287.7±5° C.

In an embodiment, crystalline Form C3 of Tafamidis may is characterized by having a DSC thermogram as shown in FIG. 9.

Optionally, the crystalline Form C3 of Tafamidis may be further characterized by data selected from the group consisting of: an X-ray powder diffraction pattern having peaks at about 5.89, 9.48, 13.74, 16.43 and 23.62±0.2 °2θ; a XRPD diffractogram as depicted in FIG. 7; a TGA pattern as depicted in FIG. 8; a DSC thermogram having a first small endothermic peak in the range of about 154.08° C.±5° C., a second endothermic peak in the range of about 160.9±5° C. and a third endothermic peak in the range of about 287.7±5° C.; a DSC pattern as depicted in FIG. 9; and combinations thereof.

Preferably the crystalline Form C3 of Tafamidis, has a crystalline purity of at least 80%, more preferably at least 90%, more preferably at least 95%, most preferably at least 99% by weight.

The invention encompasses a process for preparing the crystalline Form C3 of Tafamidis

In an embodiment process comprises, slurrying Tafamidis in a suitable first solvent; isolating the precipitated solid and drying the solid.

Preferably, Tafamidis used is Tafamidis formic acid solvate.

Preferably, solvent is a polar aprotic solvent selected from C1-C5 ketonic solvent, more preferably acetone. Preferably, solvent is chilled to −20 to −15° C.

Optionally, following the chilling step, a seeding with Tafamidis is performed to obtain crystalline Form C3 of Tafamidis. Preferably, the seeding is done with Tafamidis Form C3.

Preferably, the process further comprises a stirring step. Preferably, the stirring is for about 2 hours to about 6 hours. Preferably, the stirring is at about −20° C. to about −15° C.

Preferably, the obtained solid form is isolated. Preferably, the isolation is done by centrifugation.

The drying may be done in a vacuum oven at a temperature of about 25° C. to about 40° C. for about 1 hour to about 5 hours.

The Form C3 of Tafamidis, obtained per the present invention is substantially free from other forms of Tafamidis. “Substantially free” from other forms of Tafamidis shall be understood to mean that the polymorphs of Tafamidis contain less than 10%, preferably less than 5%, of any other forms of Tafamidis and less than 1% of other impurities.

According to a fourth aspect, the present invention provides the crystalline Tafamidis, which is herein and in the claims designated as “Form C4”, which has good flow characteristics.

The crystalline Form C4 is relatively stable towards moisture and humidity, thereby representing a crystalline form of Tafamidis, thus enhancing the efficacy of the parent molecule in lower doses.

Crystalline Form C4 of Tafamidis may be an anhydrous form or a solvated form. In specific embodiments, Form C4 of Tafamidis may be an anhydrous form or a methyl benzoate solvate.

The crystalline Form C4 according to the present invention may be characterized by powder X-ray diffraction.

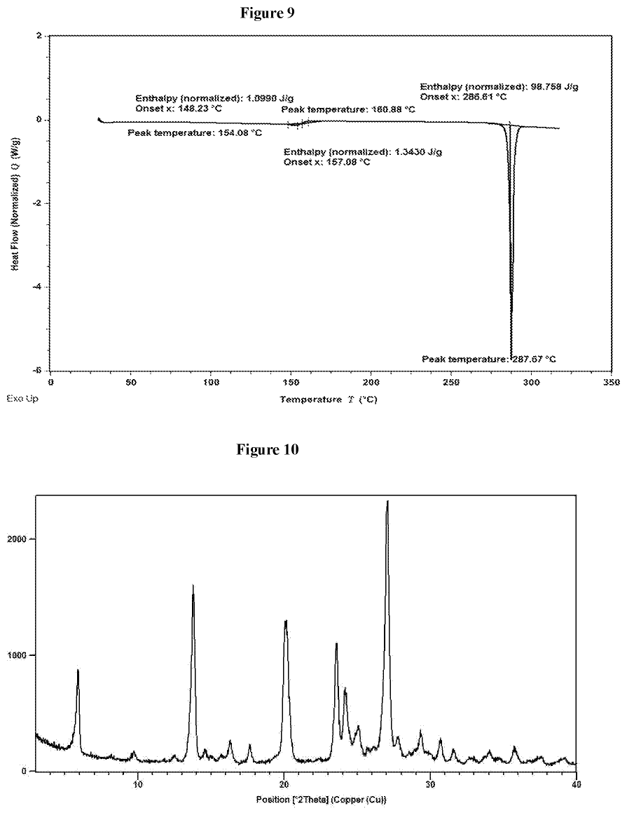

The Crystalline Form C4 may be characterized by having an XRPD diffractogram comprising peaks at 5.89, 13.72, 20.01, and 23.54±0.2 °2θ. The XRPD diffractogram may comprise further peaks at 16.28, 17.64, 24.18 and 26.97±0.2 °2θ. The XRPD diffractogram may be as depicted in FIG. 10.

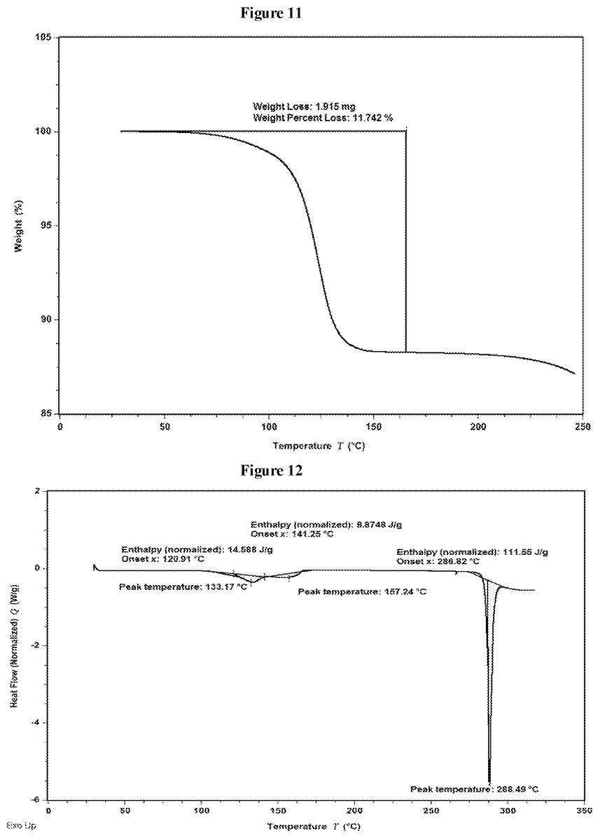

In an embodiment, crystalline Form C4 of Tafamidis is characterized by having a thermogravimetric analysis as shown in FIG. 11.

TGA data indicated a weight loss of 11.7% at temperatures up to 165° C. The TGA analysis indicates the crystalline Form C4 of Tafamidis is the methyl benzoate solvate form.

The crystalline Form C4 of Tafamidis may also be characterized as having a DSC thermogram exhibiting a first small endothermic peak with an onset at around 120.9±5° C. and a peak maximum at 133.17° C.±5° C.; a second endothermic peak with an onset at around 141.25° C.±5° C. and a peak maximum at 157.24±5° C. and a third melting endothermic peak with an onset at around 286.8±5° C. and a peak maximum at around 288.5±5° C.

In an embodiment, crystalline Form C4 of Tafamidis may is characterized by having a DSC thermogram as shown in FIG. 12.

Optionally, the crystalline Form C4 of Tafamidis may be further characterized by data selected from the group consisting of: an X-ray powder diffraction pattern having peaks at about 5.89, 13.72, 20.01, and 23.54±0.2 °2θ; an X-ray powder diffraction pattern having peaks at about 16.28, 17.64, 24.18 and 26.97±0.2 °2θ; a XRPD diffractogram as depicted in FIG. 10; a TGA pattern as depicted in FIG. 11; a DSC thermogram having a first small endothermic peak in the range of about 133.17° C.±5° C., a second endothermic peak in the range of about 157.24±5° C. and a third endothermic peak in the range of about 288.5±5° C.; a DSC pattern as depicted in FIG. 12; and combinations thereof.

Preferably the crystalline Form C4 of Tafamidis, has a crystalline purity of at least 80%, more preferably at least 90%, more preferably at least 95%, most preferably at least 99% by weight.

The invention encompasses a process for preparing the crystalline Form C4 of Tafamidis

In an embodiment process comprises, dissolving Tafamidis in methyl benzoate solvent; cooling to obtain precipitate; isolating the precipitated solid and drying the solid.

Preferably, dissolution step is done at about 50° C. to about 125° C., more preferably at about 80° C. to about 120° C. to obtain a solution.

Typically, following the heating step, the solution is cooled. Preferably, cooling is to about room temperature.

Optionally, following the cooling step, a seeding with Tafamidis is performed to obtain crystalline Form C4 of Tafamidis. Preferably, the seeding is done with Tafamidis Form C4.

Preferably, the solution is further cooled to about −10° C. to about −30° C., preferably to about −20° C. Preferably, the stirring is done for about 20 minutes to about 2 hours. Preferably, the obtained solid form is isolated. Preferably, the isolation is done by centrifugation.

The drying may be done in a vacuum oven at a temperature of about 25° C. to about 70° C. for about 5 hour to about 15 hours.

The Form C4 of Tafamidis, obtained per the present invention is substantially free from other forms of Tafamidis. “Substantially free” from other forms of Tafamidis shall be understood to mean that the polymorphs of Tafamidis contain less than 10%, preferably less than 5%, of any other forms of Tafamidis and less than 1% of other impurities.

According to a fifth aspect, the present invention provides the crystalline Tafamidis, which is herein and in the claims designated as “Form C5”, which has good flow characteristics.

The crystalline Form C5 is relatively stable towards moisture and humidity, thereby representing a crystalline form of Tafamidis, thus enhancing the efficacy of the parent molecule in lower doses.

Crystalline Form C5 of Tafamidis may be an anhydrous form or a solvated form. In specific embodiments, Form C5 of Tafamidis may be an anhydrous form or an ethyl acetate solvate.

The crystalline Form C5 according to the present invention may be characterized by powder X-ray diffraction.

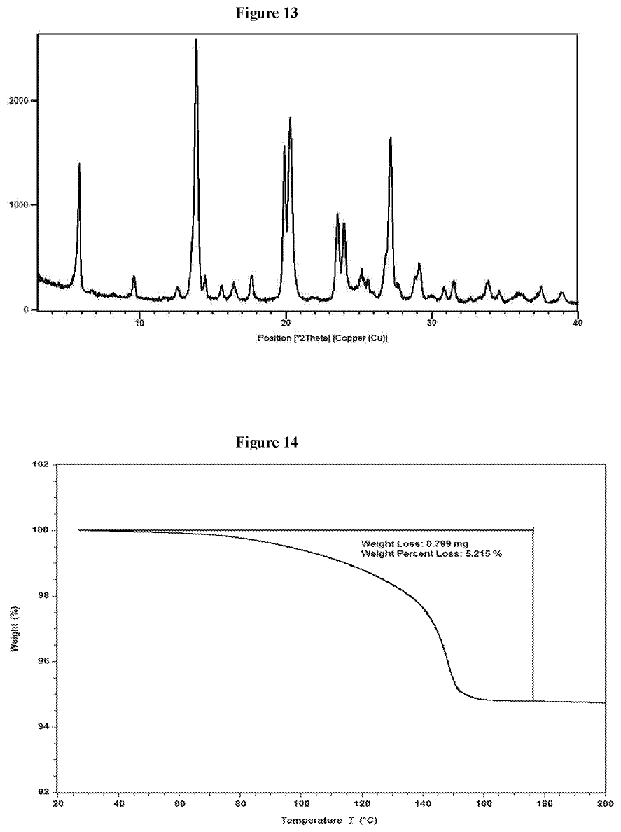

The Crystalline Form C5 may be characterized by having an XRPD diffractogram comprising peaks at 5.93, 14.01, 19.95, 20.45, 23.57 and 27.27±0.2 °2θ. The XRPD diffractogram may be as depicted in FIG. 13.

In an embodiment, crystalline Form C5 of Tafamidis is characterized by having a thermogravimetric analysis as shown in FIG. 14.

TGA data indicated a weight loss of 5.2% at temperatures up to 175° C. The TGA analysis indicates the crystalline Form C5 of Tafamidis is the ethyl acetate solvate form.

The crystalline Form C5 of Tafamidis may also be characterized as having a DSC thermogram exhibiting a first endothermic peak with an onset at around 134.9° C.±5° C. and a peak maximum at 135.65° C.±5° C. and a second melting endothermic peak with an onset at around 286.5° C.±5° C. and a peak maximum at 287.9 ±5° C.

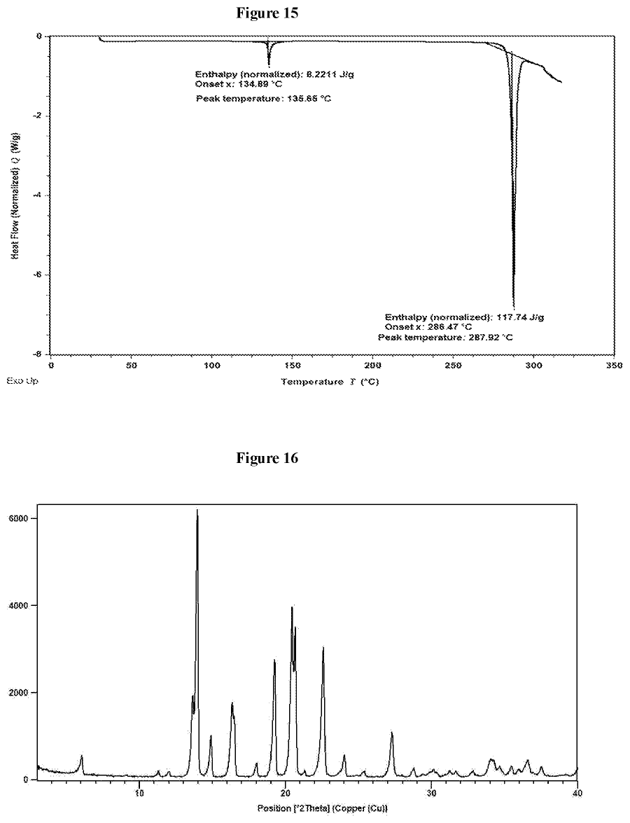

In an embodiment, crystalline Form C5 of Tafamidis may is characterized by having a DSC thermogram as shown in FIG. 15.

Optionally, the crystalline Form C5 of Tafamidis may be further characterized by data selected from the group consisting of: an X-ray powder diffraction pattern having peaks at about 5.93, 14.01, 19.95, 20.45, 23.57 and 27.27±0.2 °2θ; a XRPD diffractogram as depicted in FIG. 13; a TGA pattern as depicted in FIG. 14; a DSC thermogram having a first endothermic peak in the range of about 135.65° C.±5° C. and a second endothermic peak in the range of about 287.9±5° C.; a DSC pattern as depicted in FIG. 15; and combinations thereof.

Preferably the crystalline Form C5 of Tafamidis, has a crystalline purity of at least 80%, more preferably at least 90%, more preferably at least 95%, most preferably at least 99% by weight.

The invention encompasses a process for preparing the crystalline Form C5 of Tafamidis.

In an embodiment process comprises, slurrying Tafamidis in ethyl acetate solvent; isolating the precipitated solid and drying the solid.

Preferably, slurrying step is done at about −25° C. to about 0° C., more preferably at about −20° C. to about −10° C. to obtain a suspension.

Optionally, prior to mixing step, a seeding with Tafamidis is performed to obtain crystalline Form C5 of Tafamidis. Preferably, the seeding is done with Tafamidis Form C5.

Preferably, the stirring is done for about 5 hours to about 15 hours; more preferably for about 8 hours to about 12 hours to obtain a suspension.

Preferably, the obtained solid form is isolated. Preferably, the isolation is done by centrifugation.

The drying may be done in a vacuum oven at a temperature of about 25° C. to about 70° C. for about 1 hour to about 10 hours.

The Form C5 of Tafamidis, obtained per the present invention is substantially free from other forms of Tafamidis. “Substantially free” from other forms of Tafamidis shall be understood to mean that the polymorphs of Tafamidis contain less than 10%, preferably less than 5%, of any other forms of Tafamidis and less than 1% of other impurities.

According to a sixth aspect, the present invention provides the crystalline Tafamidis, which is herein and in the claims designated as “Form C6”, which has good flow characteristics.

The crystalline Form C6 is relatively stable towards moisture and humidity, thereby representing a crystalline form of Tafamidis, thus enhancing the efficacy of the parent molecule in lower doses.

Crystalline Form C6 of Tafamidis may be an anhydrous form or a solvated form. In specific embodiments, Form C6 of Tafamidis may be an anhydrous form or a 1,3-dioxolane solvate.

The crystalline Form C6 according to the present invention may be characterized by powder X-ray diffraction.

The Crystalline Form C6 may be characterized by having an XRPD diffractogram comprising peaks at 6.04, 13.96, 14.88, 19.23, 20.43, 20.65, 22.57 and 27.28±0.2 °2θ. The XRPD diffractogram may be as depicted in FIG. 16.

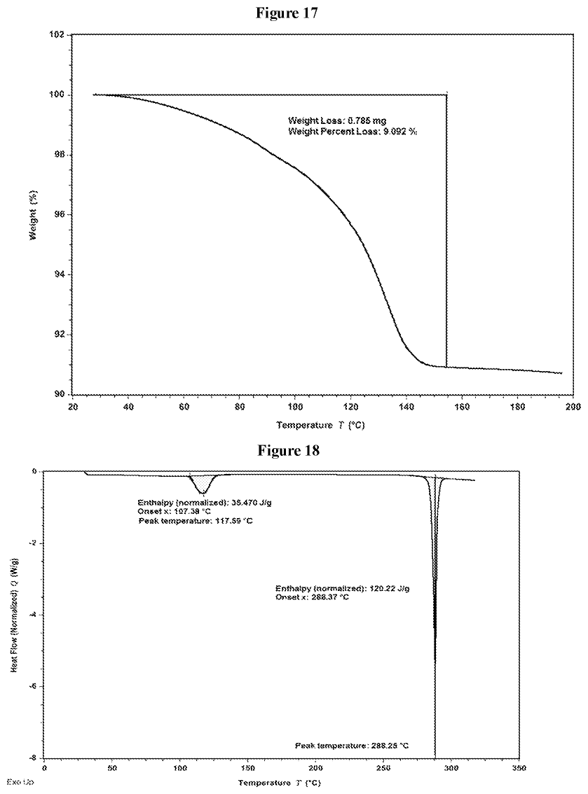

In an embodiment, crystalline Form C6 of Tafamidis is characterized by having a thermogravimetric analysis as shown in FIG. 17.

TGA data indicated a weight loss of 9.02% at temperatures up to 155° C. The TGA analysis indicates the crystalline Form C6 of Tafamidis is the 1,3-dioxolane solvate form.

The crystalline Form C6 of Tafamidis may also be characterized as having a DSC thermogram exhibiting a first endothermic peak with an onset at around 107.38° C.±5° C. and a peak maximum at 117.6° C.±5° C. and a second melting endothermic peak with an onset at around 288.37° C.±5° C. and a peak maximum at 288.25 ±5° C.

In an embodiment, crystalline Form C6 of Tafamidis may is characterized by having a DSC thermogram as shown in FIG. 18.

Optionally, the crystalline Form C6 of Tafamidis may be further characterized by data selected from the group consisting of: an X-ray powder diffraction pattern having peaks at about 6.04, 13.96, 14.88, 19.23, 20.43, 20.65, 22.57 and 27.28±0.2 °2θ; a XRPD diffractogram as depicted in FIG. 16; a TGA pattern as depicted in FIG. 17; a DSC thermogram having a first endothermic peak in the range of about 117.6° C.±5° C. and a second endothermic peak in the range of about 288.25 ±5° C.; a DSC pattern as depicted in FIG. 18; and combinations thereof.

Preferably the crystalline Form C6 of Tafamidis, has a crystalline purity of at least 80%, more preferably at least 90%, more preferably at least 95%, most preferably at least 99% by weight.

The invention encompasses a process for preparing the crystalline Form C6 of Tafamidis

In an embodiment process comprises, dissolving Tafamidis in 1,3 dioxolane solvent; evaporating the solvent to obtain precipitate; isolating the precipitated solid and drying the solid.

Preferably, dissolution step is done at about 50° C. to about 80° C., more preferably at about 55° C. to about 65° C. to obtain a solution.

Typically, following the heating step, the evaporation is done under reduced pressure.

Preferably, the obtained solid is dried. Preferably, the drying may be done in a vacuum oven at a temperature of about 55° C. to about 65° C. for about 30 minutes to about 5 hours.

The Form C6 of Tafamidis, obtained per the present invention is substantially free from other forms of Tafamidis. “Substantially free” from other forms of Tafamidis shall be understood to mean that the polymorphs of Tafamidis contain less than 10%, preferably less than 5%, of any other forms of Tafamidis and less than 1% of other impurities.

In yet another embodiment, the present invention provides, a novel pharmaceutical co-crystal comprising Tafamidis and an alkaloid component as a co-former.

The term “co-crystal” as used herein refers to crystalline materials composed of two or more different molecular and/or ionic compounds in the same crystal lattice that are associated by nonionic and noncovalent bonds, wherein at least two of the individual molecular and/or ionic compounds are solids at room temperature.

The term “co-former” refers to a compound other than Tafamidis that is also a component of the co-crystal. Thus, the co-former is part of the co-crystalline lattice. The co-former is typically a GRAS (generally regarded as safe) compound and need not exhibit any therapeutic or pharmacological activity of its own. The Registry of Toxic Effects of Chemical Substances (RTECS) database is a useful source for toxicology information, and the GRAS list maintained by the RTECS contains about 2,500 relevant compounds that may be used in the generation of one or more co-crystals.

By co-crystallizing the Tafamidis with a co-former, a new solid form is created having different properties from the Tafamidis or the conformer. For example, a co-crystal may have a different melting point, dissolution, solubility, hygroscopicity, bioavailability, toxicity, crystal morphology, density, loading volume, compressibility, physical stability, chemical stability, shelf life, taste, production costs, and/or manufacturing method than the drug.

In one embodiment co-crystal former is Nicotinamide.

According to a seventh aspect, the present invention provides a novel co-crystal of Tafamidis with Nicotinamide. The co-crystal may be in the form of a derivative thereof. The derivative may be a pharmaceutically acceptable solvate, hydrate, tautomer, anhydrate, complex, polymorph or combination thereof.

Preferably, the co-crystals comprises Tafamidis and Nicotinamide within the same crystalline phase in a molar ratio ranging from 2:1 to 1:2. More preferably the molar ratio is 1:1.

Accordingly, the co-crystal of Tafamidis with Nicotinamide is characterized by having the chemical structure as depicted in Formula (II)

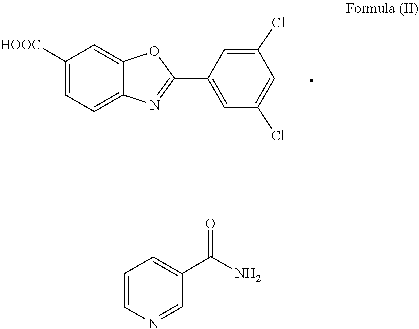 [SMILES: O=C(O)c1ccc2nc(-c3cc(Cl)cc(Cl)c3)oc2c1 ; NC(=O)c1cccnc1]

Advantageously, in some embodiments, a given percentage of the co-crystal is in crystalline form. For example, in various embodiments at least about 50% of the co-crystal is in crystalline form. In other embodiments, at least about 80 or at least about 90% of the co-crystal is in crystalline form.

In an embodiment, the co-crystal of Tafamidis with Nicotinamide can be characterized as having peaks in X-ray powder diffraction patterns obtained therefrom. For example, co-crystal can be characterized by an X-ray powder diffraction pattern having peaks at one or more of the following 2-theta diffraction angles: 7.09, 13.11, 19.68, 20.57, 21.32 and 25.30±0.2 °2θ. The XRPD diffractogram may comprise further peaks at 11.81, 17.26, 17.76, 18.62 and 28.56±0.2 °2θ.

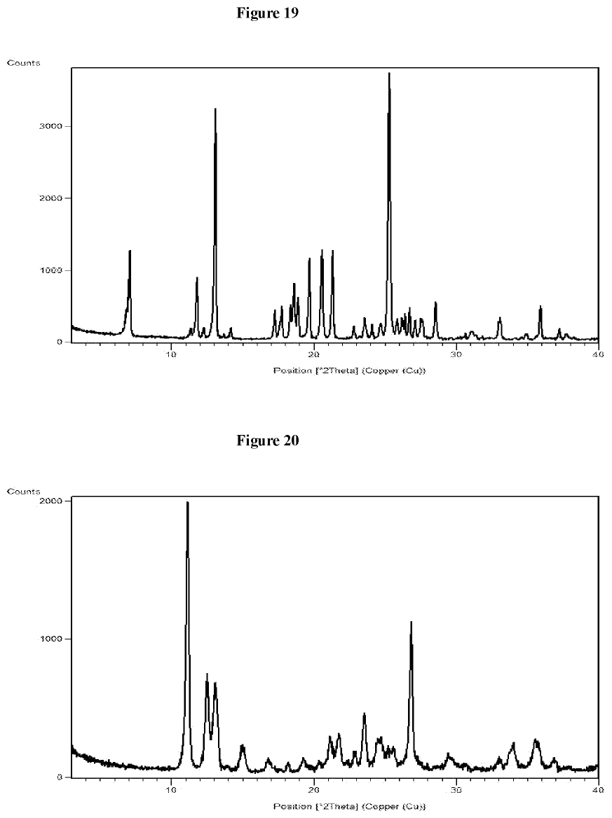

In another embodiment, the co-crystal of Tafamidis with Nicotinamide is characterized by having an XRD pattern as shown in FIG. 19.

Those skilled in the art would recognize that co-crystals of the present invention may be further characterized by a variety of other solid state spectroscopic techniques including, but not limited to, Raman spectroscopy, FTIR spectroscopy, vibrational spectroscopy, polarized light microscopy (PLM), and solid state NMR, the ¹³ C NMR and ¹H NMR (in a suitable solvent, e.g., in D₂O or DMSO-i¾) to evaluate the chemical structure, Differential Scanning Chromatography (DSC), Thermogravimetric analysis (TGA), Dynamic Gravimetric Vapor Sorption (DVS) to evaluate the hygroscopicity, hot-stage optical microscopy to examine thermal transitions and/or chromatography (e.g., HPLC) in a suitable solvent to evaluate the purity. Products as described herein can be further analysed via Karl Fischer Titration (KF) to determine the water content.

According to another aspect of the present invention, there is provided a process for preparing co-crystal of Tafamidis with Nicotinamide, the process comprising,
a. mixing Tafamidis and Nicotinamide in a suitable organic solvent;
b. stirring for sufficient time;
c. isolating the co-crystal of Tafamidis with Nicotinamide; and
d. drying.

Tafamidis base used for the above process, as well as for the following processes, may be in any polymorphic form or in a mixture of any polymorphic forms such as hydrated, solvated, non-solvated or mixture of hydrated, solvated or non-solvated forms thereof. The Tafamidis base used in the processes of the present invention can be obtained by any method known in the art, such as the one described in the U.S. Pat. No. 7,214,695 B2.

Nicotinamide used for the above process, may be in any polymorphic form or in a mixture of any polymorphic forms such as hydrated, solvated, non-solvated or mixture of hydrated, solvated or non-solvated forms thereof.

The organic solvent is preferably selected from the group comprising of polar aprotic solvent such as N,N- dimethylacetamide (DME), dimethylformamide (DMF), dimethylsulfoxide (DMSO), N-methylpyrrolidone (NMP), tetrahydrofuran (THF), sulfolane, diglyme, 1,4-dioxane and the like; ether solvent such as methyl/-butyl ether, diisoproyl ether, tetrahydrofuran (THF) and the like; ester solvent such as methyl acetate, ethyl acetate, isopropyl acetate and the like; nitrile solvent such as acetonitrile, propionitrile and the like; ketone solvent such as acetone, methyl isobutyl ketone and the like; halogenated solvent such as dichloromethane, dichloroethane, chloroform and the like; C6-C10 substituted aromatic hydrocarbons, and C1-C5 halogenated hydrocarbons; water and mixtures thereof. Preferably, organic solvent is selected from but not limited to polar aprotic solvent such as THF, DMF, DMSO and the like.

Preferably, mixing is done at about 20° C. to about 30° C.

Preferably, the process further comprises a stirring step. Preferably, the stirring is for about 5 hours to about 2 days, more preferably for about 10 hours to about 40 hours. Preferably, the stirring is done at about 20° C. to about 30° C.

Preferably, the obtained solid form is isolated. Preferably, the isolation is done by centrifugation.

The drying may be done in a vacuum oven at a temperature of about 25° C. to about 60° C., more preferably at about 30° C. to about 50° C., for about 1 hour to about 10 hours, more preferably for about 4 hours to about 8 hours.

The co-crystal of Tafamidis with Nicotinamide, obtained per the present invention is substantially free from other forms of Tafamidis. “Substantially free” from other forms of Tafamidis shall be understood to mean that the co-crystals of Tafamidis contain less than 10%, preferably less than 5%, of any other forms of Tafamidis and less than 1% of other impurities.

According to a seventh aspect, the present invention provides a novel co-crystal of Tafamidis with Caffeine. The co-crystal may be in the form of a derivative thereof. The derivative may be a pharmaceutically acceptable solvate, hydrate, tautomer, anhydrate, complex, polymorph or combination thereof.

Preferably, the co-crystal comprises Tafamidis and Caffeine within the same crystalline phase in a molar ratio ranging from 2:1 to 1:2. More preferably the molar ratio is 1:1.

Accordingly, the co-crystal of Tafamidis with Caffeine is characterized by having the chemical structure as depicted in Formula (III)

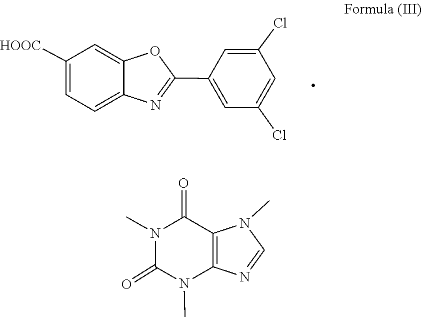

Advantageously, in some embodiments, a given percentage of the co-crystal is in crystalline form. For example, in various embodiments at least about 50% of the co-crystal is in crystalline form. In other embodiments, at least about 80 or at least about 90% of the co-crystal is in crystalline form.

In an embodiment, the co-crystal of Tafamidis with Caffeine can be characterized as having peaks in X-ray powder diffraction patterns obtained therefrom. For example, co-crystal can be characterized by an X-ray powder diffraction pattern having peaks at one or more of the following 2-theta diffraction angles: 11.16, 12.52, 13.09 21.76, 23.56 and 26.85±0.2 °2θ.

In another embodiment, the co-crystal of Tafamidis with Caffeine is characterized by having an XRD pattern as shown in FIG. 20.

According to another aspect of the present invention, there is provided a process for preparing co-crystal of Tafamidis with Caffeine, the process comprising,
a. mixing Tafamidis and Caffeine in a suitable organic solvent;
b. stirring for sufficient time;
c. isolating the co-crystal of Tafamidis with Caffeine; and
d. drying.

Caffeine used for the above process, may be in any polymorphic form or in a mixture of any polymorphic forms such as hydrated, solvated, non-solvated or mixture of hydrated, solvated or non-solvated forms thereof.

The organic solvent is preferably selected from the group comprising of polar aprotic solvent such as N,N-dimethylacetamide (DME), dimethylformamide (DMF), dimethylsulfoxide (DMSO), N-methylpyrrolidone (NMP), tetrahydrofuran (THF), sulfolane, diglyme, 1,4-dioxane and the like; ether solvent such as methyl/-butyl ether, diisoproyl ether, tetrahydrofuran (THF) and the like; ester solvent such as methyl acetate, ethyl acetate, isopropyl acetate and the like; nitrile solvent such as acetonitrile, propionitrile and the like; ketone solvent such as acetone, methyl isobutyl ketone and the like; halogenated solvent such as dichloromethane, dichloroethane, chloroform and the like; C6-C10 substituted aromatic hydrocarbons, and C1-C5 halogenated hydrocarbons; water and mixtures thereof. Preferably, organic solvent is selected from but not limited to polar aprotic solvent such as THF, DMF, DMSO and the like.

Preferably, mixing is done at about 20° C. to about 30° C.

Preferably, the process further comprises a stirring step. Preferably, the stirring is for about 5 hours to about 2 days, more preferably for about 10 hours to about 40 hours. Preferably, the stirring is done at about 20° C. to about 30° C.

Preferably, the obtained solid form is isolated. Preferably, the isolation is done by centrifugation.

The drying may be done in a vacuum oven at a temperature of about 25° C. to about 60° C., more preferably at about 30° C. to about 50° C., for about 1 hour to about 10 hours , more preferably for about 4 hours to about 8 hours.

The co-crystal of Tafamidis with Caffeine, obtained per the present invention is substantially free from other forms of Tafamidis. “Substantially free” from other forms of Tafamidis shall be understood to mean that the co-crystals of Tafamidis contain less than 10%, preferably less than 5%, of any other forms of Tafamidis and less than 1% of other impurities.

The process of invention may be used as a method for purifying any form as well as for preparing other solid state forms of Tafamidis, or solid state forms thereof, as well as other Tafamidis salts or solid state forms thereof. The process includes preparing the solid state forms of the present invention, and converting it to other solid state forms of Tafamidis. Alternatively, the process includes preparing the solid state form of the present invention , and converting it to Tafamidis salt. The conversion can be done, for example, by a process including reacting the novel Tafamidis form with an appropriate acid or base.

Acid salts include hydrochloride, hydrobromide, hydroiodide, nitrate, sulfate, phosphate, acetate, adipate, alginate, aspartate, benzoate, benzenesulfonate, bisulfate, butyrate, citrate, camphorate, camphor sulfonate, cyclopentane propionate, digluconate, dodecylsulfate, methanesulfonate, ethanesulfonate, 2-naphthalenesulfonate fumarate, glucoheptanoate, sulfate glycerophosphate, hemisulfate, heptanoate, hexanoate, 2-hydroxyethanesulfonate, lactate, maleate, nicotinate, oxalate, tartrate, malate, maleate, pamoate, pectinate, persulfate, 3-phenyl-propionate, picrate, pivalate, propionate, succinate, thiocyanate, tosylate and undecanoate and the like.

Base salts include ammonium salts, meglumine salt, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and the like.

According to another aspect of the present invention, there is provided a pharmaceutical composition comprising polymorphic forms of Tafamidis or co-crystals as described above, together with one or more pharmaceutically acceptable excipients. The Tafamidis used in the preparation of pharmaceutical compositions may substantially consist of one of forms C1, C2, C3, C4, C5, C6 or co-crystals, described above, or may substantially consist of a combination of two or more of said forms.

According to yet another aspect of the present invention there is provided use of Tafamidis as described above, in the preparation of a medicament useful in treating or preventing cardiomyopathy of wild type or hereditary transthyretin-mediated amyloidosis.

The invention will now be further described by the following examples, which are illustrative rather than limiting.

### EXAMPLES

### Example 1: Process for the Preparation of Tafamidis Form C1

To a chilled solution of Methylene dichloride (200 ml) was added Tafamidis Form C1 seed (163 mg) at −20 to −15° C. The solution was stirred for 5-10 min. Tafamidis formic acid solvate form (10 g) was added into the above chilled solvent and maintained at −20 to −15° C. for 5 hours. The temperature increased to 0-10° C. and continued to stir at this temperature for 4.30 hours. The temperature was further increased to 25° C. and stirred the reaction mass for 8 hours. The solids were isolated by filtration and dried under vacuum at 40° C. for 1.30 hours to yield 7.8 gm of title compound.

The 7.8 gm of resulted material further slurried in 78 ml of acetone at −10° C. for 3 hours. The solids were isolated by filtration and dried under vacuum at 35° C. for 1 hour and then at

60-65° C. for about 40 hours to yield 6 gm of the title compound. The solid was analyzed by XRD, TGA and DSC and identified as Form C1 as depicted in FIGS. 1 to 3.

### Example 2: Process for the Preparation of Tafamidis Form C1

To a chilled solution of Methylene dichloride (5 ml) and 0.2 ml of HC1 (36%) was added Tafamidis meglumine salt (0.5 g) at -5 to 0° C. The reaction mass was maintained at −5 to 0° C. for 4 hours. The solid were isolated by filtration to yield 0.368 gm white colour solid.

The resultant solids were further slurried in water at 0-5° C. for 1 hour, filtered and dried under vacuum at 35° C. for 1 hour to yield 0.25 gm of the title compound.

The solid was analyzed by XRD, TGA and DSC and identified as Form C1 as depicted in FIGS. 1 to 3.

### Example 3: Process for the Preparation Tafamidis Form C2

To a chilled solution of ethanol (50 ml) was added Tafamidis formic acid solvate form (5 g) at −15 to −20° C. The reaction mass was maintained at −15 to −20° C. for 3 hours. Added prechilled solution of n-Heptane (25 ml) to the above reaction mass at −15 to −20° C. and continued the stirring for 45 mins. The solids were isolated by filtration and dried under vacuum at 40° C. for 2 hours to yield 3.4 gm of the title compound.

The solid was analyzed by XRD, TGA and DSC and identified as Form C2 as depicted in FIGS. 4 to 6.

### Example 4: Process for the Preparation of Tafamidis form C3

To a chilled solution of Acetone (50 ml) was added Tafamidis formic acid solvate form (5 g) at −15 to −20° C. The reaction mass was maintained at same temperature for 3 hours. The solids were isolated by filtration and dried under vacuum at 25 to 30° C. for 1-2 hours to yield 4.2 gm of the title compound.

The solid was analyzed by XRD, TGA and DSC and identified as Form C3 as depicted in FIGS. 7 to 9.

### Example 5: Process for the Preparation Tafamidis Form C4

Tafamidis (1 g) was dissolved in Methyl benzoate (70 ml) at 120° C. The clear solution was cooled to room temperature. The solution was further chilled to −20° C. and maintained for 20 min. The solids were isolated by filtration and dried under vacuum at 50° C. for 8 hours to yield 0.85 gm of the title compound.

The solid was analyzed by XRD, TGA and DSC and identified as Form C4 as depicted in FIGS. 10 to 12.

### Example 6: Process for the Preparation Tafamidis Form C5

To a chilled solution of Ethyl acetate (30 ml) was added Tafamidis Form C4 (2 g) at −20-to-15° C. The reaction mass was maintained at −10 to −20° C. for about 8 hours. The solids were isolated by filtration and dried under vacuum at 40° C. for 2 hours to yield 1.5 gm of the title compound.

The solid was analyzed by XRD, TGA and DSC and identified as Form C5 as depicted in FIGS. 13 to 15.

### Example 7: Process for the Preparation Tafamidis Form C6

Tafamidis (0.5 g) was dissolved in 1,3-Dioxolane (45 ml) at 65° C. The solvent was removed under reduced pressure using rotavapor at 65° C. and dried for 30 min to yield 0.45 gm of the title compound.

The solid was analyzed by XRD, TGA and DSC and identified as Form C6 as depicted in FIGS. 16 to 18.

### Example 8: Process for the Preparation of Tafamidis Nicotinamide Co-Crystal

Tafamidis (2.0 gm) and Nicotinamide (0.792 gm) were suspended in THF (40 ml) and the suspension was stirred at 20 to 25° C. for 22.5 hours. The solids were isolated by filtration and dried under vacuum at 40° C. for 6 hours to yield 1.8 gm of the title compound.

H-NMR reveals a molar ratio of Tafamidis to Nicotinamide of about 1:1. The crystallinity was confirmed by powder X-ray diffraction pattern as depicted in FIG. 19.

### Example 9: Process for the Preparation of Tafamidis Caffeine Co-Crystal

Tafamidis (2.0 gm) and Caffeine (1.26 gm) were suspended in THF (40 ml) and the suspension was stirred at 20 to 25° C. for 22.5 hours. The solids were isolated by filtration and dried under vacuum at 40° C. for 6 hours to yield 1.9 gm of the title compound.

H-NMR reveals a molar ratio of Tafamidis to Caffeine of about 1:1. The crystallinity was confirmed by powder X-ray diffraction pattern as depicted in FIG. 20.

### Example 10: Process for the Preparation of Tafamidis Form C1

Dissolved Tafamidis (100 gm) in a solution of Methylene dichloride (1500 ml) and triethyl amine (50 ml) at 20 to 25° C. The resulted clear solution treated with charcoal for 15-20 minutes, followed by filtration through hyflo bed to remove charcoal, and the hyflo bed was washed with Methylene dichloride (200 ml). The combined filtrate transferred into another clean RBF, fallowed by addition of IPA HCl (130 ml) and Tafamidis Form C1 seed slurry (4 gm in 40 ml Methylene dichloride) at 23±3° C. over a period of about 30-60 min.

The reaction mass was stirred further at 27±3° C. for 24-48 hours. The solids were isolated by filtration to yield 130-180 gm of the wet Tafamidis form C1. The resulted wet solids were further slurried twice in Methylene dichloride (2×100 ml) to remove traces of TEA salts. The wet material obtained after Methylene dichloride purifications, was dried first at ambient temperature in VTD under vacuum for 2 hours, then at 40±3° C. for 4 hours, then at 50±3° C. for 8 hours. The resulted material co-milled with 1 mm mesh, followed by micronization and dried in VTD under vacuum first at ambient temperature for 2 hours, at 40±3° C. for 6 hours, 50±3° C. for 10 hours and finally at 50±3° C. for 8 hours to yield 60-70 gm of the title compound.

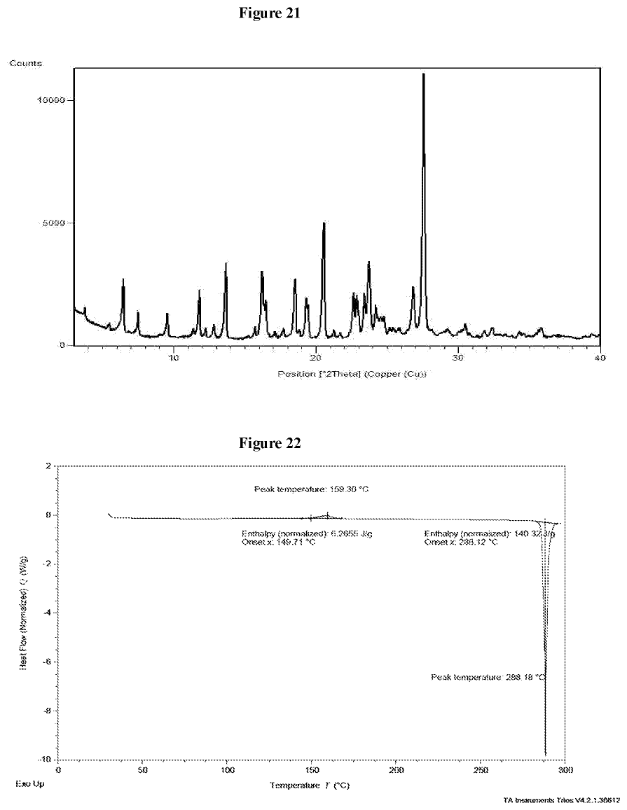

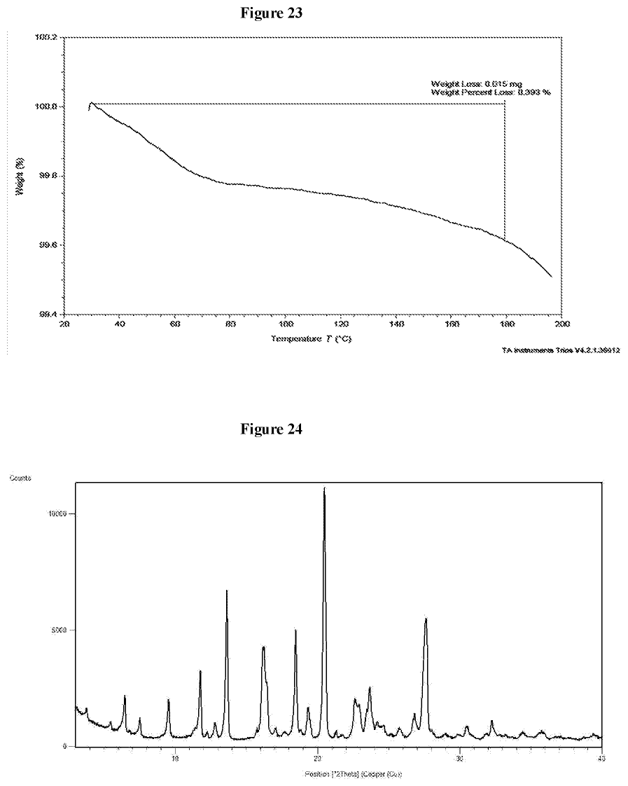

The solid was analyzed by XRD, DSC and TGA and identified as Form C1 as depicted in FIGS. 21 to 23.

### Example 11: Process for the Preparation of Tafamidis Form C1

Dissolved Tafamidis (100 gm) in a solution of Methylene dichloride (1500 ml) and triethyl amine (50 ml) at 20 to 25° C. The resulted clear solution treated with charcoal for 15-20 minutes, followed by filtration through hyflo bed to remove charcoal, and the hyflo bed was washed with Methylene dichloride (200 ml). The combined filtrate transferred into another clean RBF, followed by addition of IPA HCl (130 ml) and Tafamidis Form C1 seed slurry (4 gm in 40 ml Methylene dichloride) at 23±3° C. over a period of about 30-60 min. The reaction mass was stirred further at 27±3° C. for 24-48 hours. The solids were isolated by filtration to yield 130-180 gm of the wet Tafamidis form C1. The resulted wet solids were further slurried in Methylene dichloride (1000 ml) to remove traces of TEA salts. The wet material obtained after Methylene dichloride purifications, was further slurried in Acetone (1000 ml) at −2 ±2° C. for 30-60 min. The solids were isolated by filtration and dried first at 40° C. under vacuum for 4 hours and, then at 90° C. for 20 hours to yield 80-90 gm of the title compound.

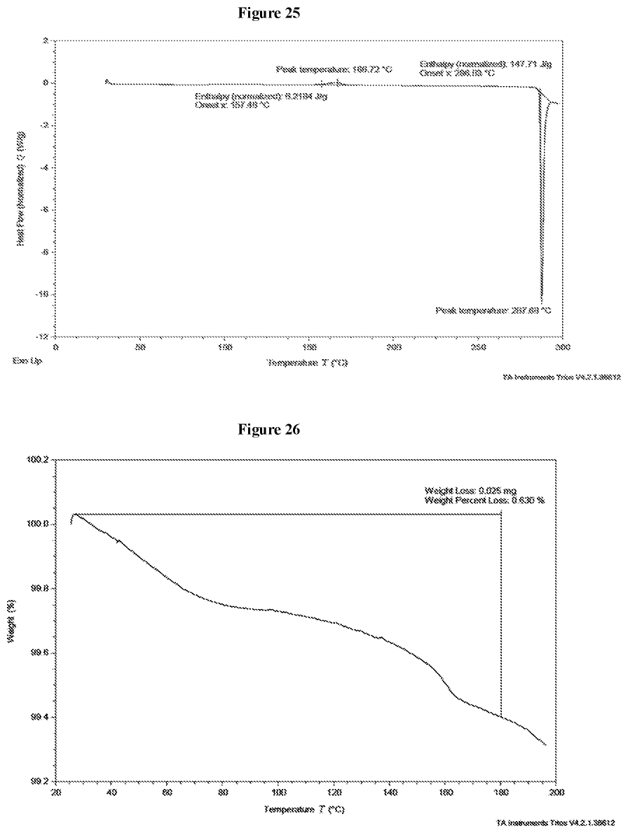

The solid was analyzed by XRD, DSC and TGA and identified as Form C1 as depicted in FIGS. 24 to 26.

1. Crystalline Form C1 of Tafamidis.

2. The Crystalline Form C1 of Tafamidis of claim 1, characterized by XRPD diffractogram with characteristics peaks at 5.48, 6.44, 7.51, 9.57, 11.80, 13.65, 18.51 and 20.53±0.2 °2θ.

3. The Crystalline Form C1 of Tafamidis of claim 2, further characterized by XRPD diffractogram with characteristics peaks at 16.24, 19.36, 23.68 and 27.55±0.2 °2θ.

4. (canceled)

5. (canceled)

6. The Crystalline Form C1 of Tafamidis of claim 1, characterized by a DSC thermogram having a small endotherm onset at around 152.63±5° C. and an onset melting point at around 288.5±5° C. °C.

7. (canceled)

8. The Crystalline Form C1 of Tafamidis of claim 1, further characterized by data selected from the group consisting of:
an X-ray powder diffraction pattern having peaks at about 5.48, 6.44, 7.51, 9.57, 11.80, 13.65, 18.51 and 20.53±0.2 °2θ;
an X-ray powder diffraction pattern having peaks at about 16.24, 19.36, 23.68 and 27.55±0.2 °2θ;
a DSC thermogram having a first endothermic peak in the range of about 161.44±5° C. and a second endothermic peak in the range of about 288.5±5° C.;
a TGA pattern indicating a weight loss of 0.8% at temperatures up to 170° C.; and
combinations thereof.

9. A process for preparing crystalline Form C1 of Tafamidis of claim 1, the process comprising the steps of:
a) slurrying Tafamidis in a suitable non polar solvent;
b) stirring for sufficient time,
c) isolating the solid; and
d) drying the solid.

10. The process for preparing the Crystalline Form C1 of Tafamidis of claim 9, further comprising:
a1) seeding the solution from step (a) with Tafamidis Form C1 at −20° C. to −15° C. and allowing the solution to stir until a slurry forms.

11. The process for preparing the Crystalline Form C1 of Tafamidis of claim 9, wherein the non polar solvent is halogenated solvent selected from the group comprising of dichloromethane, dichloroethane, chloroform and the like.

12. A process for preparing crystalline Form C1 of Tafamidis of claim 1, the process comprising the steps of:
a) slurrying Tafamidis salt in a suitable mixture of non polar solvent and acid;
b) stirring for sufficient time,
c) isolating the solid; and
d) drying the solid.

13. The process for preparing the Crystalline Form C1 of Tafamidis of claim 12, wherein Tafamidis salt is Tafamidis meglumine.

14. The process for preparing the Crystalline Form C1 of Tafamidis of claim 12, wherein the non polar solvent is halogenated solvent selected from the group comprising of dichloromethane, dichloroethane, chloroform and the like.

15. The process for preparing the Crystalline Form C1 of Tafamidis of claim 12, wherein the acid is aqueous hydrochloric acid.

16. A process for preparing crystalline Form C1 of Tafamidis of claim 1, the process comprising the steps of:
a) treating Tafamidis with a suitable base in a suitable non polar solvent;
b) treating Tafamidis salt solution with IPA-HCl;
c) isolating the solid; and
d) drying the solid.
wherein the process is carried out without isolating Tafamidis salt.

17. The process for preparing the Crystalline Form C1 of Tafamidis of claim 16, further comprising:
b1) seeding the IPA-HCl solution first with Tafamidis Form C1 at 20° C. to 25° C. and allowing the solution to stir until a slurry forms.

18. The process for preparing the Crystalline Form C1 of Tafamidis of claim 16, wherein the base is selected from an inorganic or organic base.

19. The process for preparing the Crystalline Form C1 of Tafamidis of claim 18, wherein the base is triethylamine.

20. The process for preparing the Crystalline Form C1 of Tafamidis of claim 16, wherein the non polar solvent is halogenated solvent selected from the group comprising of dichloromethane, dichloroethane, chloroform and the like.

21. A pharmaceutical composition in the form of a solid, liquid, powder, elixir, injectable solution and the like, comprising the Crystalline Form C1 of Tafamidis according to claim 1 and a pharmaceutically acceptable excipient.

22. A pharmaceutical composition of claim 21, wherein Crystalline Form C1 of Tafamidis is formulated into tablets, film-coated tablets, sugar coated tablets, capsules, soft gelatin capsules, hard gelatin capsules, troches, aqueous suspensions or solutions, dispersions, injectables and other pharmaceutical forms.

23. A method of prevention and/or treatment of transthyretin-mediated amyloidosis comprising administering to a patient in need thereof a therapeutically effective amount of crystalline Form C1 of Tafamidis according to claim 1.

24. (canceled)

25. (canceled)
